(19) United States
(12) Patent Application Publication
(10) Pub. No.: US 2019/0216707 A1
(43) Pub. Date: Jul. 18, 2019

(54) COMPOSITION FOR PREVENTING HAIR LOSS AND PROMOTING HAIR GROWTH, COMPRISING PHYTOESTROGEN AS AN ACTIVE INGREDIENT

(71) Applicant: STEMORE CO., LTD., Incheon (KR)

(72) Inventors: Jong Hyuk SUNG, Seongnam-si (KR); Seung Hyun KIM, Incheon (KR)

(73) Assignee: STEMORE CO., LTD., Incheon (KR)

(21) Appl. No.: 16/361,779

(22) Filed: Mar. 22, 2019

Related U.S. Application Data
(63) Continuation of application No. PCT/KR2017/010659, filed on Sep. 27, 2017.

(30) Foreign Application Priority Data
Sep. 27, 2016 (KR) 10-2016-0123803

(51) Int. Cl. A61K 8/49; A61K 31/352; A61K 36/48; A61K 8/9789; A61Q 7/00

(52) U.S. Cl. CPC A61K 8/498; A61K 31/352; A61Q 7/00; A61K 8/9789; A61K 36/48

(57) ABSTRACT

The present invention relates to a composition for preventing hair loss and promoting hair growth, which comprises phytoestrogen as an active ingredient and, more particularly, to a pharmacological composition, a cosmetic composition, and a health functional food composition, for preventing hair loss and promoting hair growth, the composition comprising, as an active ingredient, phytoestrogen, which is an estrogen structural analogue derived from a leguminous plant.

### CROSS-REFERENCE TO RELATED APPLICATION

This application is a continuation of International Application No. PCT/KR2017/010659 filed on Sep. 27, 2017, which claims priority to and the benefit of Korean Patent Application No. 10-2016-0123803, filed on Sep. 27, 2016, the disclosure of which is incorporated herein by reference in its entirety.

### TECHNICAL FIELD

The present invention relates to a composition for preventing hair loss and promoting hair growth, which includes a phytoestrogen as an active ingredient.

### BACKGROUND ART

Human hair primarily acts as a defense function to protect the scalp from external stimuli such as ultraviolet rays and the like, and plays an aesthetic function expressing the external appearance of an individual. In modern society, however, hair loss is frequently caused by environmental pollution, natural factors such as strong ultraviolet rays, and the like, and physiological factors such as stress, hormonal imbalance, and the like, and unlike the past when hair loss was confined to adult males, hair loss occurs regardless of sex or age, and therefore, there is an urgent need for a material for the prevention and treatment of hair loss.

Hair follicles, which are a physiological organ in the body that produces hair, is involved in the growth, maintenance, and elimination of hair, and particularly, the hair cycle, which is divided into the anagen phase where hair is produced during postnatal development and the produced hair actively grows, the catagen phase where hair degenerates, the telogen phase where hair is maintained until it is eliminated, and the exogen phase where hair loss occurs, is repeated, thus enabling the growth and maintenance of hair. As hair loss has increased and demand for hair growth and hair growth promotion has increased, interest in the activity of hair follicles has recently increased. The activity of hair follicles is caused by dermal papilla cells, and in particular, the proliferation and differentiation of dermal papilla cells are involved primarily in the progression of the hair growth cycle and hair formation. In the anagen phase where hair grows actively, dermal papilla cells actively proliferate and are actively differentiated, the growth of hair is stopped, and these cells die in the catagen phase, the telogen phase, and the exogen phase where hair loss occurs.

Therefore, hair growth and hair loss are closely related to the proliferation and death of dermal papilla cells, and thus for the alleviation and treatment of hair loss, methods of prolonging the anagen phase by inducing the proliferation of these cells, or inhibiting apoptosis and shortening the catagen, telogen, and exogen phases are attracting attention. In addition, cell division and migration in the vicinity of the dermal papilla is closely related to the growth of hair, and hair is newly generated from the dermal papilla in the anagen phase, and cells are activated by various cytokines, hormones, and the like to be transferred to the dermal papilla, thereby affecting hair growth.

Currently, representative drugs approved by the FDA and enabling hair growth and hair growth promotion are minoxidil and finasteride. However, these drugs are accompanied by side effects such as hair growth on the whole body, sexual dysfunction, and the like, and the specific mechanism of action of minoxidil has not been clearly identified. Therefore, efforts have been made to develop a drug that promotes the proliferation of dermal papilla cells in the hair follicles, increases the expression of growth factors, and is harmless to the human body.

### DISCLOSURE

### Technical Problem

An object of the present invention is to provide a pharmaceutical composition for preventing hair loss or promoting hair growth, which includes a phytoestrogen as an active ingredient, and the like.

However, technical problems to be solved by the present invention are not limited to the above-described technical problems, and other unmentioned technical problems will become apparent from the following description to those of ordinary skill in the art.

### Technical Solution

The present invention provides a pharmaceutical composition for preventing hair loss or promoting hair growth, which includes, as an active ingredient, one or more selected from phytoestrogens represented by Formulae 1 to 4 below:

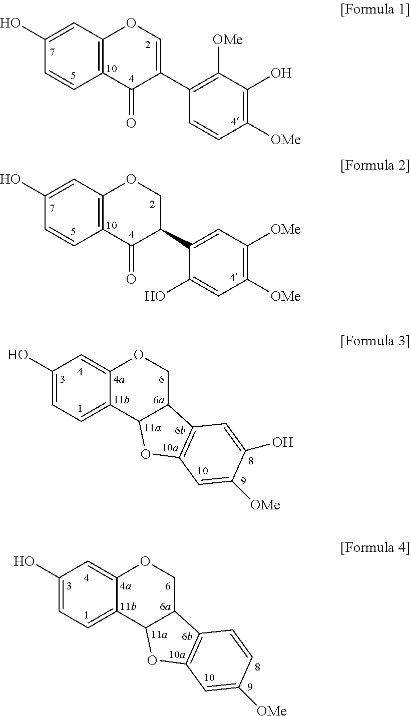 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

The phytoestrogens may be derived from a leguminous plant.

The phytoestrogens may increase the survival rate of human hair follicle dermal papilla cells.

The phytoestrogens may have an activity of elongating human hair length.

The phytoestrogens may be included in the composition at a concentration of 0.005 μM to 1 mM.

The pharmaceutical composition may be a preparation selected from the group consisting of a cream, a gel, a patch, a spray, an ointment, a plaster, a lotion, a liniment, a paste, and a cataplasma, which are dermatologic agents.

According to an embodiment of the present invention, there is provided a cosmetic composition for preventing hair loss or promoting hair growth, which includes, as an active ingredient, one or more selected from phytoestrogens represented by Formulae 1 to 4 below:

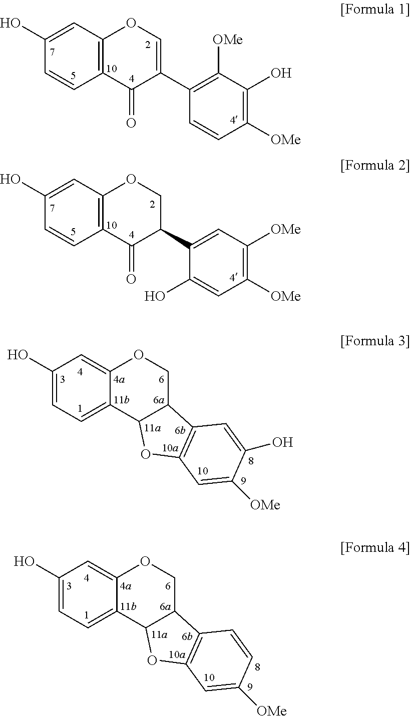 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

The phytoestrogens may be derived from a leguminous plant.

The cosmetic composition may be formulated into a hair tonic, a hair lotion, a hair cream, a hair spray, a hair mousse, a hair gel, a hair conditioner, a hair shampoo, a hair rinse, a hair pack, a hair treatment, an eyebrow growth agent, an eyelash growth agent, an eyelash nutrient, a shampoo for a pet, or a rinse for a pet.

According to another embodiment of the present invention, there is provided a health functional food composition for preventing hair loss or promoting hair growth, which includes, as an active ingredient, one or more selected from phytoestrogens represented by Formulae 1 to 4 below:

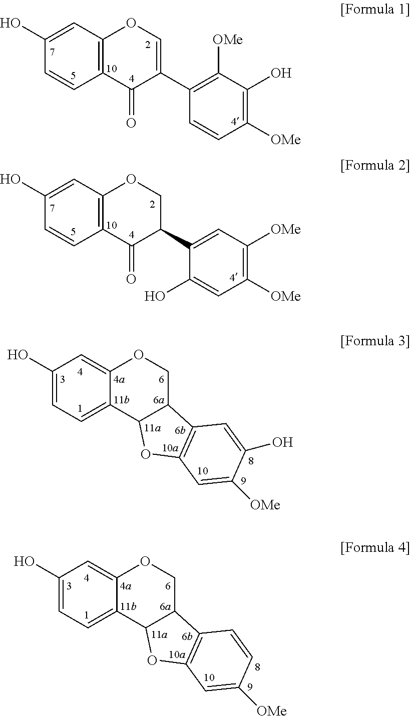 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

The phytoestrogens may be derived from a leguminous plant.

According to another embodiment of the present invention, there is provided a method of preventing or treating hair loss, or promoting hair growth, which includes administering a composition including, as an active ingredient, one or more selected from phytoestrogens represented by Formulae 1 to 4 below:

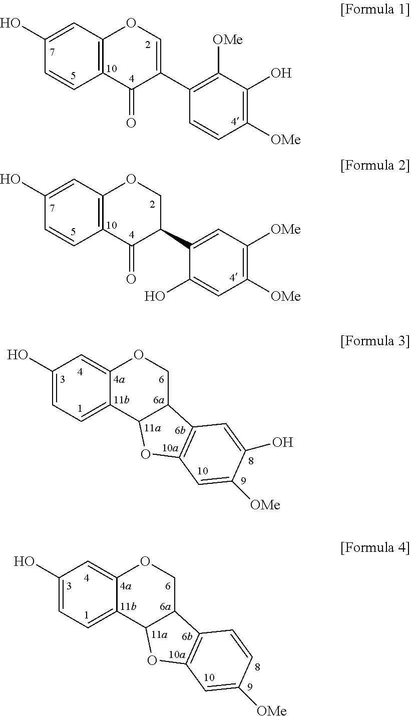 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

According to another embodiment of the present invention, there is provided a use of a composition including, as an active ingredient, one or more selected from phytoestrogens represented by Formulae 1 to 4 below, for preparing an agent for preventing or treating hair loss, or promoting hair growth:

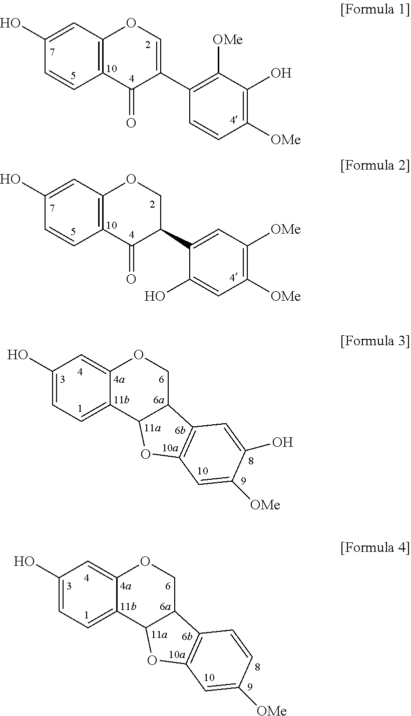 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

### Advantageous Effects

The present invention relates to a composition for preventing hair loss or promoting hair growth, which includes a phytoestrogen as an active ingredient, and the phytoestrogen can exhibit an effect of promoting the proliferation of immortalized dermal papilla cells and promoting the expression of growth factors associated with hair tissue differentiation, and significantly exhibit an effect of promoting hair growth on the hair-removed back of a mouse, and thus can be used for preventing hair loss or promoting hair growth.

### DESCRIPTION OF DRAWINGS

FIGS. 1A to 1D are graphs showing results of confirming an effect of promoting the proliferation of immortalized dermal papilla cells (I-DPCs) according to concentrations of phytoestrogens and a control (minoxidil) over time, and respectively show the cases of Formulae 1 to 4.

FIG. 2 is a set of images showing results of confirming an effect of promoting hair growth on the back of mice when phytoestrogens were administered to the mice via subcutaneous injection.

FIG. 3 is a graph showing an increase in hair weight in accordance with hair growth promotion when phytoestrogens were administered via subcutaneous injection.

### BEST MODE

As described above, in line with an increase in hair loss due to stress in addition to hormone secretion changes, there has been an increasing need for a pharmaceutical composition capable of preventing hair loss and/or promoting hair growth, but among compounds known to be safe in the human body, few compounds capable of effectively preventing hair loss and/or promoting hair growth have been reported. Therefore, the inventors of the present invention had verified that phytoestrogens could exhibit an effect of promoting the proliferation of immortalized dermal papilla cells and promoting the expression of hair tissue differentiation-associated growth factors, and significantly exhibit an effect of promoting hair growth on the hair-removed back of a mouse, and thus completed the present invention.

Hereinafter, the present invention will be described in more detail.

The present invention provides a pharmaceutical composition for preventing hair loss or promoting hair growth, which includes, as an active ingredient, one or more selected from phytoestrogens represented by Formulae 1 to 4 below:

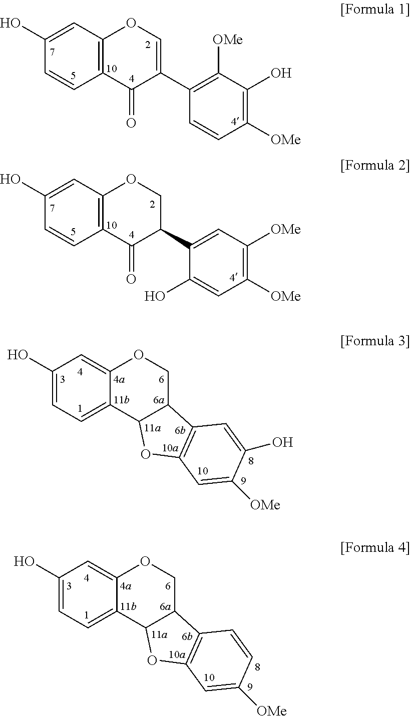 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

The term “phytoestrogen” as used herein refers to a non-steroidal compound present in plants exhibiting an estrogenic effect in the body of an animal, and in particular, phytoestrogens are relatively abundantly included in soybean processed foods including soybeans (soybean milk, tofu, soybean paste, and the like), various cereals or fruits, alfalfa, clover, and the like. The activity of the vegetable estrogens is only 1/100 to 1/1,000 of that of endogenous estrogens produced in the body, but the content is much higher so that a sufficient estrogenic effect can be obtained. The phytoestrogens of the present invention may be derived from “Dalbergia Oliveri”, which is a leguminous plant, but the present invention is not limited thereto.

The term “hair loss prevention” or “hair growth promotion” as used herein is intended to include another term “hair growth” or “hair growth promotion” used in the art.

In one embodiment of the present invention, the inventors of the present invention selected phytoestrogens, which are estrogen structural analogues derived from the leguminous plant Dalbergia Oliveri and promote the proliferation of dermal papilla cells, and selected the compounds of Formulae 1 to 4 from among various phytoestrogenic active ingredients.

The phytoestrogens may increase the survival rate of human hair follicle dermal papilla cells and have an activity of elongating human hair length.

In addition, the phytoestrogen may be included in the composition at a concentration of 0.005 μM to 1 mM, preferably 1 μM to 10 μM, but the present invention is limited thereto. In this regard, when the concentration of the phytoestrogen is less than the above concentration range, an effect of preventing hair loss and promoting hair growth is insignificant, and when the concentration of the phytoestrogen exceeds the above concentration range, cytotoxicity occurs.

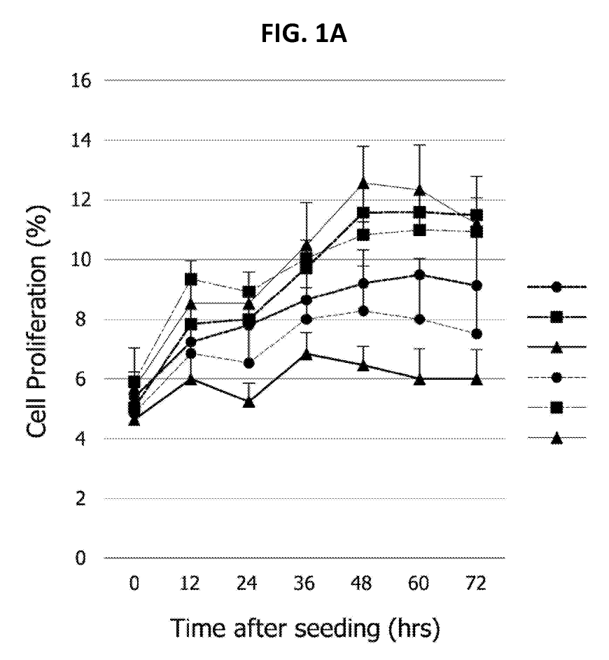

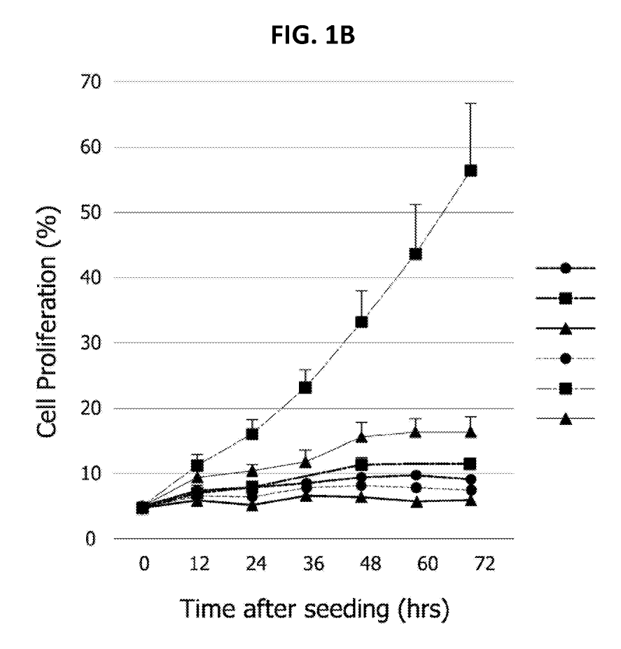

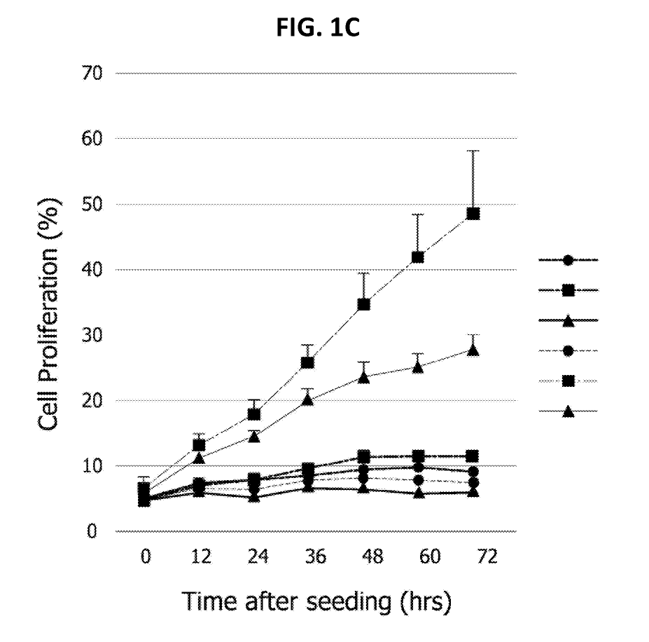

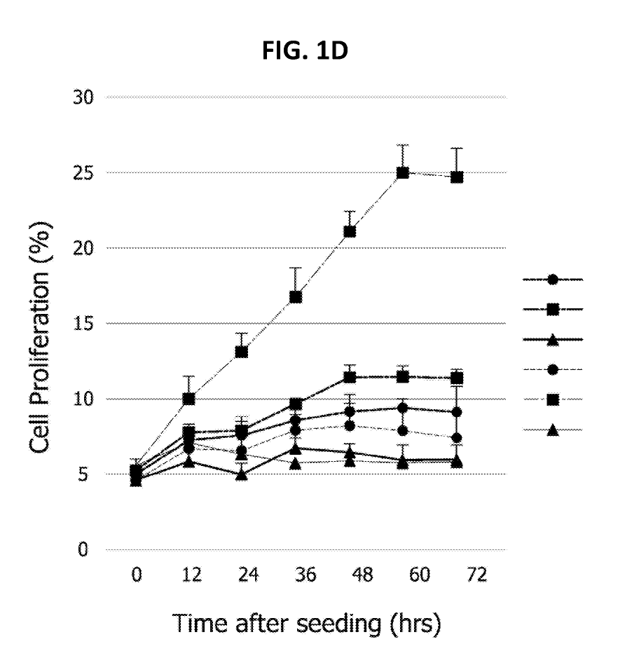

In one embodiment of the present invention, as a result of examining an effect of the phytoestrogen compounds of Formulae 1 to 4 on dermal papilla cells, it was confirmed that the phytoestrogen compounds were able to significantly promote the proliferation of immortalized dermal papilla cells (I-DPCs) at a concentration of 1 μM to 10 μM (see FIGS. 1A to 1D).

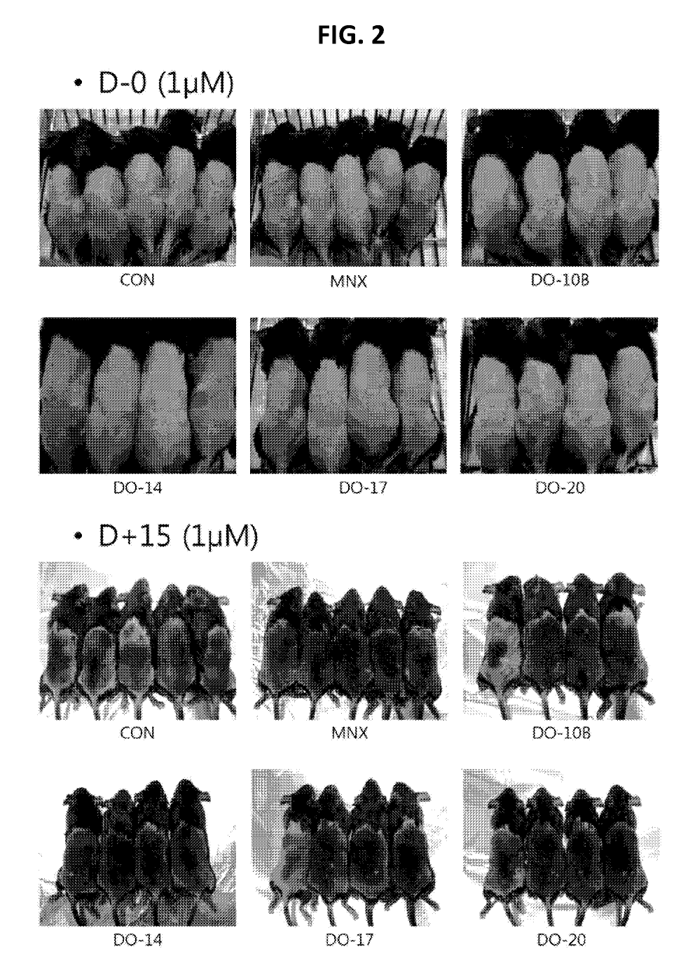

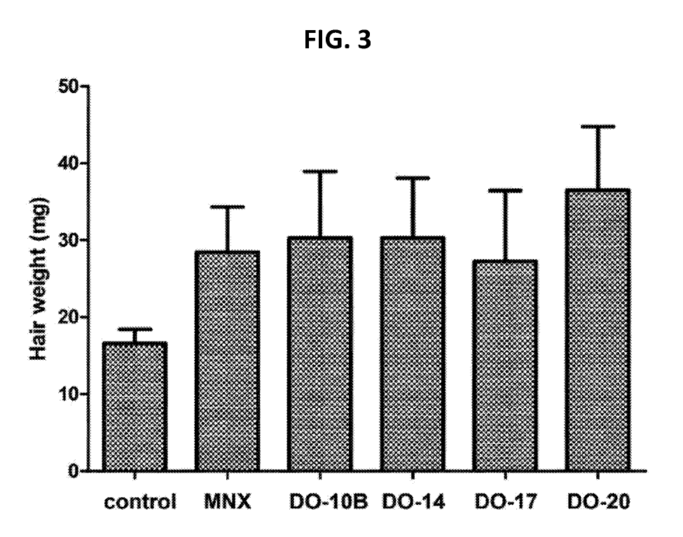

In addition, as a result of examining whether the phytoestrogen compounds of Formulae 1 to 4 are able to exhibit an effect of promoting hair growth in vivo, it was confirmed that when each compound was percutaneously administered by application on the hair-removed back of a mouse or subcutaneously injected thereinto, these groups exhibited significantly increased hair growth as compared to the non-administered control and minoxidil (see FIGS. 2 and 3). Accordingly, the compounds of the present invention are more harmless than conventional synthetic compositions, and may exhibit an effect of promoting the proliferation of dermal papilla cells, which are core cells that play a role in hair growth, and promoting the expression of hair tissue differentiation-associated growth factors, and may significantly exhibit an effect of promoting hair growth on the hair-removed back of a mouse, and thus may be effectively used as a composition for preventing hair loss and promoting hair growth.

The phytoestrogens are preferably a dermatologic composition, and a dermatological pharmaceutical composition including the phytoestrogen compound of the present invention as an active ingredient may be a dermatologic agent for preventing hair loss and alleviating and promoting hair growth and may be formulated into the form of a dermatologic agent such as a cream, a gel, a patch, a spray, an ointment, a plaster, a lotion, a liniment, a paste, or a cataplasma, but the present invention is not limited thereto.

The compounds of the present invention may be used in the form of a pharmaceutically acceptable salt, and the salt may be an acid addition salt formed by a pharmaceutically acceptable free acid. The acid addition salt is obtained from: inorganic acids such as hydrochloric acid, nitric acid, phosphoric acid, sulfuric acid, hydrobromic acid, hydriodic acid, nitrous acid, and phosphorous acid; or nontoxic organic acids such as aliphatic mono- and dicarboxylates, phenyl-substituted alkanoates, hydroxy alkanoates and alkandioates, aromatic acids, and aliphatic and aromatic sulfonic acids. Examples of these pharmaceutically nontoxic salts include sulfates, pyrosulfates, bisulfates, sulfites, bisulfites, nitrates, phosphates, monohydrogen phosphates, dihydrogen phosphates, methaphosphates, pyrophosphate chlorides, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, butyne-1,4-dioates, hexane-1,6-dioates, benzoates, chlorobenzoates, methylbenzoates, dinitro benzoates, hydroxybenzoates, methoxybenzoates, phthalates, terephthalates, benzene sulfonates, toluene sulfonates, chlorobenzene sulfonates, xylenesulfonates, phenyl acetates, phenylpropionates, phenylbutyrates, citrates, lactates, β-hydroxybutyrates, glycolates, maleates, tartrates, methanesulfonates, propanesulfonates, naphthalene-1-sulfonates, naphthalene-2-sulfonates, and mandelates.

Acid addition salts according to the present invention may be prepared using a conventional method, for example, by dissolving the compounds of Formulae 1 to 4 in an excess of an aqueous acid solution and precipitating the salt using a water-miscible organic solvent, e.g., methanol, ethanol, acetone, or acetonitrile. The acid addition salts may also be prepared by heating the same amounts of the compounds of Formulae 1 to 4 and an acid or alcohol in water, and then evaporating and drying the resulting mixture or suction-filtering the precipitated salt.

In addition, pharmaceutically acceptable metallic salts may be prepared using bases. Alkali metal or alkaline earth metal salts are obtained by, for example, dissolving a compound in an excess of an alkali metal hydroxide or alkaline earth metal hydroxide solution, filtering a non-soluble compound salt, and evaporating and drying the filtrate. At this time, it is pharmaceutically preferable that a sodium salt, a potassium salt, or a calcium salt is prepared as a metal salt. In addition, silver salts corresponding thereto are obtained by reacting an alkali metal or an alkaline earth metal salt with a suitable silver salt (e.g., silver nitrate).

Moreover, the compounds of Formulae 1 to 4 of the present invention includes not only pharmaceutically acceptable salts thereof, but also all salts, hydrates, and solvates that may be prepared using general methods.

Addition salts according to the present invention may be prepared using general methods, for example, by dissolving the compounds of Formulae 1 to 4 in a water-miscible organic solvent, e.g., acetone, methanol, ethanol, acetonitrile, or the like, adding an excess of an organic acid or an aqueous inorganic acid solution thereto, and then precipitating or crystallizing the resulting solution. Subsequently, the solvent or the excess of the acid may be evaporated from the mixture, followed by drying, thereby obtaining an addition salt, or the precipitated salt may be subjected to suction filtration.

In a case in which the composition of the present invention is used as a drug, the pharmaceutical composition including the compounds of Formulae 1 to 4 or pharmaceutically acceptable salts thereof as active ingredients may be formulated into various dosage forms for oral or parenteral administration in clinical trials and administered, but the present invention is not limited thereto.

Examples of preparations for oral administration include tablets, pills, hard/soft capsules, liquids, suspensions, emulsions, syrups, granules, elixirs, and the like. These preparations include, in addition to the active ingredient, a diluent (e.g., lactose, dextrose, sucrose, mannitol, sorbitol, cellulose, and/or glycine), and a lubricant (e.g., silica, talc, stearic acid and magnesium or calcium salts thereof, and/or polyethylene glycol). Tablets may include a binder such as magnesium aluminum silicate, starch paste, gelatin, methyl cellulose, sodium carboxymethylcellulose, and/or polyvinyl pyrrolidone, and in some cases, may include a disintegrating agent such as starch, agar, alginic acid or sodium salts thereof, or a boiling mixture and/or an absorbent, a coloring agent, a flavoring agent, and a sweetening agent.

The pharmaceutical composition including the compounds of Formulae 1 to 4 or pharmaceutically acceptable salts thereof as active ingredients, according to the present invention may be administered parenterally, and parenteral administration is performed via subcutaneous injection, intravenous injection, intramuscular injection, or intrathoracic injection. In this regard, to formulate preparations for parenteral administration, the pharmaceutical composition including the compounds of Formulae 1 to 4 or pharmaceutically acceptable salts thereof may be mixed with a stabilizer or a buffer in water to prepare a solution or a suspension, followed by preparation into an ampoule or vial unit dosage form. The composition may be sterilized and/or include an adjuvant such as a preservative, a stabilizer, wettable powder, an emulsifying agent, a salt for the control of osmotic pressure, and/or a buffer, and other therapeutically effective materials, and may be formulated using a conventional method, such as mixing, granulation, or coating.

In addition, a dose of the composition of the present invention, which is to be administered to the human body, may vary depending on the age, body weight, and gender of patients, administration forms, health conditions, and the severity of diseases, and may generally range from 0.001 mg/day to 1,000 mg/day, preferably 0.01 mg/day to 500 mg/day, with respect to an adult patient with a body weight of 60 kg, and the composition of the present invention may also be administered once a day or in multiple doses at regular intervals in accordance with the prescription of a doctor or a pharmacist.

The present invention also provides a cosmetic composition for preventing hair loss or promoting hair growth, which includes, as an active ingredient, one or more selected from phytoestrogens represented by Formulae 1 to 4 below. A detailed description of the phytoestrogens is the same as provided above:

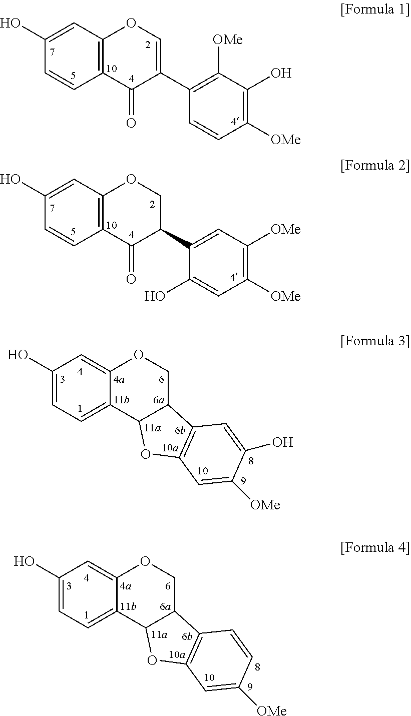 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

When the compounds of Formulae 1 to 4 of the present invention are used as active ingredients of a quasi-drug composition, the compounds of Formulae 1 to 4 that exhibit an effect of preventing hair loss and promoting hair growth may be added as they are, or may be used in combination with other quasi-drugs or quasi-drug ingredients, and may be appropriately used according to a general method. Mixing amounts of the active ingredients may be appropriately determined according to the purpose of use.

The quasi-drug composition for preventing hair loss and promoting hair growth of the present invention is not particularly limited in terms of its preparation, and may be variously formulated into the form of quasi-drugs known in the art that exhibit an effect of preventing hair loss and promoting hair growth. The formulated quasi-drugs include a hair tonic, a hair lotion, a hair cream, a hair spray, a hair mousse, a hair gel, a hair conditioner, a hair shampoo, a hair rinse, a hair pack, a hair treatment, an eyebrow growth agent, an eyelash growth agent, an eyelash nutrient, a shampoo for a pet, a rinse for a pet, a hand cleaner, a detergent soap, a soap, a disinfectant cleaner, wet tissue, a mask, an ointment, a patch, a filter filler, and the like, and include all quasi-drugs in a general sense.

In addition, for each preparation, the quasi-drug composition for preventing hair loss and promoting hair growth may be mixed with other ingredients arbitrarily selected according to the preparations of other quasi-drugs, the purpose of use, or the like. Mixing amounts of the active ingredients may be appropriately determined according to the purpose of use, and the composition may include, for example, general adjuvants such as a thickener, a stabilizer, a solubilizer, vitamins, a pigment, and a flavor, a carrier, and the like.

The amount of the active ingredients may range from 0.0001 wt % to 10 wt % with respect to a total weight of the composition. When the amount of the active ingredients is greater than 10 wt %, color and stability deteriorate in preparation of the composition, and when the amount of the active ingredients is less than 0.0001 wt %, effects thereof are insignificant. The quasi-drug composition including the compounds of Formulae 1 to 4 as active ingredients of the present invention has little cytotoxicity and almost no side effects on cells as confirmed in the therapeutic index thereof, and thus may be effectively used as a quasi-drug material.

The present invention also provides a health functional food composition for preventing hair loss or promoting hair growth, which includes, as an active ingredient, one or more selected from phytoestrogens represented by Formulae 1 to 4 below. A detailed description of the phytoestrogens is the same as provided above:

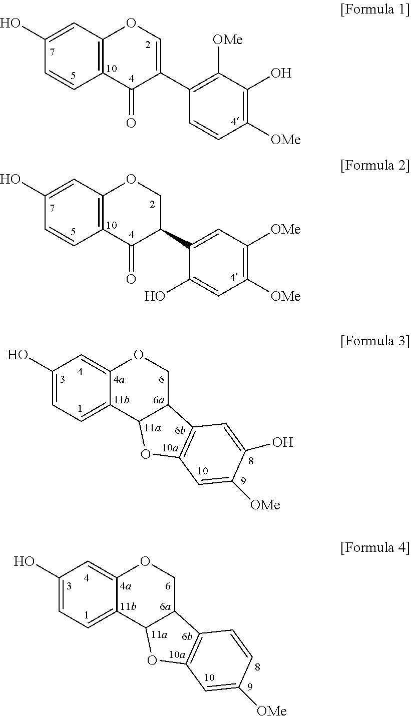 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

The food composition according to the present invention may be prepared into various forms according to a general method known in the art. The food composition may be prepared by adding the compounds of Formulae 1 to 4 of the present invention to a general food, without being limited thereto, such as beverages (including alcoholic beverages), fruits and processed foods thereof (e.g., canned fruits, bottled foods, jam, marmalade, and the like), fish, meat and processed foods thereof (e.g., ham, sausage, corn, beef, and the like), bread and noodles (e.g., Japanese-style noodles, buckwheat noodles, ramen, spaghetti, macaroni, and the like), fruit juices, various drinks, cookies, taffy, dairy products (e.g., butter, cheese, and the like), edible vegetable oils, margarine, vegetable proteins, retort foods, frozen foods, various seasonings (e.g., soybean paste, soy sauce, other sauces, and the like), and the like. In addition, a nutritional supplement may be prepared by adding the compounds of Formulae 1 to 4 of the present invention into the form of capsules, tablets, pills, or the like, but the present invention is not limited thereto. In addition, as a health functional food, for example, the compounds of Formulae 1 to 4 of the present invention may be prepared into the form of tea, juice, and drink, and then may be liquefied, granulated, encapsulated, and powdered so that the food can be ingested as a health drink, but the present invention is not limited thereto. In addition, the compounds of Formulae 1 to 4 may be prepared into the form of powder or concentrate to be used as food additives. In addition, the compounds of Formulae 1 to 4 of the present invention may be mixed with active ingredients known to be effective in preventing hair loss and promoting hair growth to prepare a composition.

When the compounds of Formulae 1 to 4 of the present invention are used as health drinks, the health drink composition may include various flavor enhancers, natural carbohydrates, or the like as additional ingredients as in general beverages. Examples of the above-described natural carbohydrates include monosaccharides such as glucose and fructose; disaccharides such as maltose and sucrose; polysaccharides such as cyclodextrin; and sugar alcohols such as xylitol, sorbitol, erythritol, and the like. Sweeteners include natural sweeteners such as thaumatin and stevia extracts; and synthetic sweeteners such as saccharin and aspartame. The proportion of the natural carbohydrates generally ranges from about 0.01 g to about 0.04 g, preferably about 0.02 g to about 0.03 g, with respect to 100 mL of the composition of the present invention.

In addition, the compounds of Formulae 1 to 4 of the present invention may be included as active ingredients of a health functional food for preventing hair loss and promoting hair growth, and the amount thereof is an effective amount for achieving an effect of preventing hair loss and promoting hair growth and may range from, but is not limited to, 0.01 wt % to 100 wt % with respect to a total weight of the composition. The food composition of the present invention may be prepared by mixing the compounds of Formulae 1 to 4 with other active ingredients known to be effective in preventing hair loss and promoting hair growth.

In addition to the above-listed ingredients, the health food of the present invention may include various nutritional supplements, vitamins, electrolytes, flavors, colorants, pectic acid and salts thereof, alginic acid and salts thereof, organic acids, a protective colloid thickener, a pH adjuster, a stabilizer, a preservative, glycerin, alcohols, a carbonating agent, and the like. In addition, the health food of the present invention may include flesh for the preparation of natural fruit juice, fruit juice beverage, and vegetable beverages. These ingredients may be used alone or a mixture thereof may be used. The proportion of these additives is not very important, but the amounts of the additives generally range from 0.01 part by weight to 0.1 part by weight with respect to 100 parts by weight of the composition of the present invention.

Hereinafter, the present invention will be described in detail with reference to the following examples. However, these examples are provided for illustrative purposes only and are not intended to limit the scope of the present invention.

### EXAMPLES

### Example 1. Isolation of Phytoestrogen Compounds Having Effect of Promoting Proliferation of Dermal Papilla Cells (DPCs)

A ground portion including the leaves and twigs of dried Dalbergia oliveri was ultrasonically extracted by repeating 3 times with 100% methanol each for 4 hours, and then the extract was filtered and then concentrated under reduced pressure using a vacuum concentrator to obtain a methanol crude extract. The crude extract was suspended in distilled water, and then sequentially fractionated with n-hexane and ethyl acetate (EtOAc) to obtain respective fractions. The EtOAc fractions were separated into small fractions by silica gel column chromatography (mobile phase:hexane-acetone 40:1→1:1). These small fractions were confirmed by TLC, and the small fractions in which an isoflavonoid was identified were again subjected to silica gel column chromatography (mobile phase:chloroform-methanol 50:1→5:1) and further divided into smaller fractions. These smaller fractions were further subjected to YMC column chromatography (mobile phase:mixed solvent of water and methanol) to obtain DO-10B, DO-14, DO-17, and DO-20.

As a result of performing 1D NMR, 2D NMR, and MS spectroscopic analyses on the above compounds, the compounds were confirmed as violanone for DO-10B, (3R)-5′-methoxyvestiol for DO-14, (6aR, 11aR)-3,8-dihydroxy-9-methoxypterocarpan for DO-17, and 3-hydroxy-9-methoxypterocarpan for DO-20. These isolated phytoestrogen compounds DO-10B, DO-14, DO-17, and DO-20 were respectively represented by Formulae 1 to 4.

### Example 2. Evaluation of Biological Function of Phytoestrogen Compounds

2-1. Cell Culture

Human-derived immortalized dermal papilla cells (I-DPCs; donated by Professor Sungyoung Kwan of the College of Medicine at Kyungpook National University; Shin, Seung-Hyun, et al. “Establishment and characterization of an immortalized human dermal papilla cell line.” BMB reports 44.8 (2011): 512-516.) were cultured in Follicle Dermal Papilla Cell Growth Medium supplemented with 10% fetal bovine serum (PBS) and penicillin-streptomycin antibiotics for 3 days to prepare a dermal papilla cell line. On day 3 of culture, the cells cultured in the culture dish were transferred to a 96-well plate, and when the cells were attached to the bottom of each well, each well was treated with DO-10B, DO-14, DO-17, and DO-20 at a concentration of 10 μM and cultured for 48 hours.

2-2. Confirmation of Effect of Phytoestrogen Compounds on Proliferation of Dermal Papilla Cells

Minoxidil is an oral vasodilating drug developed in the United States and mainly used for severe hypertension, but an effect thereof as a hair loss therapeutic agent has recently been confirmed and thus it is used as an agent for external application. In the present example, effects of minoxidil, and the compounds DO-10B, DO-14, DO-17, and DO-20 on the proliferation of dermal papilla cells were compared with each other.

In particular, the I-DP cell line was prepared by culturing under the same conditions as in Example <2-1>, and the cells were treated with DO-10B, DO-14, DO-17, and DO-20 at a concentration of 0.1 μM, 1 μM, or 10 μM in a cell culture medium, and then cultured for a total of 72 hours. At 0, 12, 24, 36, 48, 60, and 72 hours after the start of culture, the degree of saturation of cells attached to the bottom of each well (phase object confluence, %) was confirmed using the Incucyte System™.

As a result, as illustrated in FIGS. 1A to 1D, it was confirmed that DO-10B, DO-14, DO-17, and DO-20 affected the proliferation of I-DPCs in a concentration-dependent manner. In particular, it was confirmed that upon treatment with these compounds at a concentration of 1 μM to 10 μM, the proliferation of I-DPCs was significantly promoted (see FIGS. 1A to 1D).

2-3. Confirmation of Effect of Phytoestrogens on Hair Growth Promotion

Since it was confirmed that DO-10B, DO-14, DO-17, and DO-20 promoted the proliferation of dermal papilla cells, it was examined whether these compounds are able to exhibit an effect of actually promoting hair growth through induction of the anagen phase of the hair cycle from the telogen phase when administered in vivo.

In particular, to confirm the effects of DO-10B, DO-14, DO-17, and DO-20 on hair growth promotion when applied and subcutaneously administered, 6-week-old male C3H/HeNCrljOri mice were purchased and divided into a negative control, a positive control (minoxidil-treated group), and groups treated with DO-10B, DO-14, DO-17, and DO-20 to be used for experiments. To confirm a hair growth effect in the mice, when the mice became 6 weeks of age, first, the hair of the back of each mouse was removed using a clipper.

To confirm the effects of DO-10B, DO-14, DO-17, and DO-20 on hair growth promotion when subcutaneously injected, PBS containing 1 μM DO-10B, DO-14, DO-17, and DO-20 was subcutaneously injected into the hair-removed backs of mice at a daily cycle of 2 to 3 times, and the mice were raised for a total of 2 weeks. After the end of the rearing period, the hair of the back of each mouse was removed again using a mouse clipper, and the weight of the collected hair was measured to compare the hair weights of the non-administered group and the minoxidil-administered group.

As a result, as illustrated in FIG. 2, it was confirmed that, when DO-10B, DO-14, DO-17, and DO-20 were subcutaneously injected, the hair cycle was induced to the anagen phase by DO-10B, DO-14, DO-17, and DO-20 and it was visually confirmed that these groups exhibited significant hair growth as compared to that of the non-administered group (see FIG. 2). In addition, it was confirmed that the hair weights of these groups were about two-fold or more that of the non-administered control. In addition, it was confirmed that the hair weights of these groups exhibited levels equal to or greater than that of the minoxidil-administered control (see FIG. 3).

Therefore, it may be confirmed that hair growth is promoted by DO-10B, DO-14, DO-17, and DO-20, and these compounds exhibit a hair growth effect that is equal to or greater than that of minoxidil, which is a representative drug enabling hair growth and hair growth promotion.

Preparation examples of compositions including the compounds of the present invention will now be described, but these examples are provided for illustrative purposes only and are not intended to limit the scope of the present invention.

### Preparation Example 1: Preparation of Powder

| | |
|---|---|
| Phytoestrogen | 20 mg |
| Lactose hydrate | 100 mg |
| Talc | 10 mg |

The above ingredients were mixed and airtight packages were filled therewith, thereby completing the preparation of powder.

### Preparation Example 2: Preparation of Tablets

| | |
|---|---|
| Phytoestrogen | 10 mg |
| Corn starch | 100 mg |
| Lactose hydrate | 100 mg |
| Magnesium stearate | 2 mg |

The above ingredients were mixed, and then tablets were prepared according to a general method of preparing tablets.

### Preparation Example 3: Preparation of Capsules

| | |
|---|---|
| Phytoestrogen | 10 mg |
| Microcrystalline cellulose | 3 mg |
| Lactose hydrate | 14.8 mg |
| Magnesium stearate | 0.2 mg |

The above ingredients were mixed, and then gelatin capsules were filled therewith according to a general method of preparing capsules, thereby completing the preparation of capsules.

### Preparation Example 4: Preparation of Injections

| | |
|---|---|
| Phytoestrogen | 10 mg |
| Mannitol | 180 mg |
| Sterile distilled water for injection | 2,974 mg |
| Sodium monohydrogenphosphate | 26 mg |

The above ingredients were mixed, and then ampoules were prepared according to a general method of preparing an injection such that the above ingredients were contained in a single ampoule (2 ml).

### Preparation Example 5: Preparation of Liquids

| | |
|---|---|
| Phytoestrogen | 10 mg |
| Isomerized sugar | 10 mg |
| Mannitol | 5 mg |
| Purified water | appropriate amount |
| Lemon flavor | appropriate amount |

Each ingredient was added to and dissolved in purified water according to a general method of preparing a liquid, a lemon flavor was added in an appropriate amount, the above ingredients were mixed, and purified water was added thereto such that a total amount of the resulting solution was adjusted to 100 ml, and after sterilization, a brown bottle was filled therewith, thereby completing the preparation of liquids.

### Preparation Example 6: Preparation of Health Functional Food

| Phytoestrogen | 10 | mg |
|---|---|---|
| Vitamin mixture | appropriate amount | |
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinic acid amide | 1.7 | mg |
| Folic acid | 50 | μg |
| Calcium pantothenate | 0.5 | mg |
| Mineral mixture | appropriate amount | |
| Ferrous sulfate | 1.75 | mg |
| Zinc oxide | 0.82 | mg |
| Magnesium carbonate | 25.3 | mg |
| Potassium monophosphate | 15 | mg |
| Dicalcium phosphate | 55 | mg |
| Potassium citrate | 30 | mg |
| Calcium carbonate | 100 | mg |
| Magnesium chloride | 24.8 | mg |

Although ingredients relatively suitable for health foods are mixed in the above-described mixing ratio of vitamins and minerals as an exemplary embodiment, the mixing ratio may be arbitrarily modified, and the above ingredients may be mixed according to a general method of preparing health foods to prepare granules, and the granules may be used in the preparation of a health food composition according to a general method.

### Preparation Example 7: Preparation of Health Drinks

| | | |
|---|---|---|
| Phytoestrogen | 10 | mg |
| Vitamin C | 15 | g |
| Vitamin E (powder) | 100 | g |
| Ferrous lactate | 19.75 | g |
| Zinc oxide | 3.5 | g |
| Nicotinic acid amide | 3.5 | g |
| Vitamin A | 0.2 | g |
| Vitamin B₁ | 0.25 | g |
| Vitamin B₂ | 0.3 | g |

| | |
|---|---|
| Purified water | appropriate amount |

The above-listed ingredients were mixed according to a general method of preparing a health drink, the mixture was heated and stirred at 85 □ for about 1 hour to prepare a solution, the solution was filtered, the filtrate was collected in 21 sterilized containers and then sealed and sterilized, followed by refrigerated storage, which were then used for the preparation of a health drink composition of the present invention.

Although ingredients relatively suitable for use in favorite beverages are mixed in the above-described composition ratio as an exemplary example, the mixing ratio may be arbitrarily varied depending on local and national preferences such as demand classes, demand countries, purposes of use, and the like.

Preparation examples of cosmetic compositions including the compounds of the present invention will now be described, but these examples are provided for illustrative purposes only and are not intended to limit the scope of the present invention.

### Preparation Example 1: Nourishing Lotion (Milk Lotion)

| | |
|---|---|
| Phytoestrogen | 2.0 wt % |
| Squalane | 5.0 wt % |
| Cera alba | 4.0 wt % |
| Polysorbate 60 | 1.5 wt % |
| Sorbitan sesquioleate | 1.5 wt % |
| Liquid paraffin | 0.5 wt % |
| Caprylic/capric triglyceride | 5.0 wt % |
| Glycerin | 3.0 wt % |
| Butylene glycol | 3.0 wt % |
| Propylene glycol | 3.0 wt % |
| Carboxyvinyl polymer | 0.1 wt % |
| Triethanolamine | 0.2 wt % |
| Preservative, pigment, flavor | appropriate amounts |
| Purified water to 100 wt % | |

Although ingredients relatively suitable for nourishing lotions are mixed in the above-described composition ratio as an exemplary example, the mixing ratio may be arbitrarily modified, and the nourishing lotion may be prepared according to a general preparation method in the cosmetic field.

### Preparation Example 2: Skin Softener (Skin Lotion)

| | |
|---|---|
| Phytoestrogen | 2.0 wt % |
| Glycerin | 3.0 wt % |
| Butylene glycol | 2.0 wt % |
| Propylene glycol | 2.0 wt % |
| Carboxyvinyl polymer | 0.1 wt % |
| PEG 12 nonyl phenyl ether | 0.2 wt % |
| Polysorbate 80 | 0.4 wt % |
| Ethanol | 10.0 wt % |
| Triethanolamine | 0.1 wt % |
| Preservative, pigment, flavor | appropriate amounts |
| Purified water to 100 wt % | |

Although ingredients relatively suitable for skin softeners are mixed in the above-described composition ratio as an exemplary example, the mixing ratio may be arbitrarily modified, and the skin softener may be prepared according to a general preparation method in the cosmetic field.

### Preparation Example 3: Nourishing Cream

| | |
|---|---|
| Phytoestrogen | 2.0 wt % |
| Polysorbate 60 | 1.5 wt % |
| Sorbitan sesquioleate | 0.5 wt % |
| PEG60 hardened castor oil | 2.0 wt % |
| Liquid paraffin | 10 wt % |
| Squalane | 5.0 wt % |
| Caprylic/capric triglyceride | 5.0 wt % |
| Glycerin | 5.0 wt % |
| Butylene glycol | 3.0 wt % |
| Propylene glycol | 3.0 wt % |
| Triethanolamine | 0.2 wt % |
| Preservative | appropriate amount |
| Pigment | appropriate amount |
| Flavor | appropriate amount |
| Purified water to 100 wt % | |

Although ingredients relatively suitable for nourishing creams are mixed in the above-described composition ratio as an exemplary example, the mixing ratio may be arbitrarily modified, and the nourishing cream may be prepared according to a general preparation method in the cosmetic field.

### Preparation Example 4: Massage Cream

| | |
|---|---|
| Phytoestrogen | 1.0 wt % |
| Cera alba | 10.0 wt % |
| Polysorbate 60 | 1.5 wt % |
| PEG60 hardened castor oil | 2.0 wt % |
| Sorbitan sesquioleate | 0.8 wt % |
| Liquid paraffin | 40.0 wt % |
| Squalane | 5.0 wt % |
| Caprylic/capric triglyceride | 4.0 wt % |
| Glycerin | 5.0 wt % |
| Butylene glycol | 3.0 wt % |
| Propylene glycol | 3.0 wt % |
| Triethanolamine | 0.2 wt % |
| Preservative, pigment, flavor | appropriate amounts |
| Purified water to 100 wt % | |

Although ingredients relatively suitable for massage creams are mixed in the above-described composition ratio as an exemplary example, the mixing ratio may be arbitrarily modified, and the massage cream may be prepared according to a general preparation method in the cosmetic field.

### Preparation Example 5: Pack

| | |
|---|---|
| Phytoestrogen | 1.0 wt % |
| Polyvinyl alcohol | 13.0 wt % |
| Sodium carboxymethylcellulose | 0.2 wt % |
| Glycerin | 5.0 wt % |
| Allantoin | 0.1 wt % |
| Ethanol | 6.0 wt % |
| PEG 12 nonyl phenyl ether | 0.3 wt % |
| Polysorbate 60 | 0.3 wt % |
| Preservative, pigment, flavor | appropriate amounts |
| Purified water to 100 wt % | |

Although ingredients relatively suitable for packs are mixed in the above-described mixing ratio as an exemplary example, the mixing ratio may be arbitrarily modified, and the pack may be prepared according to a general preparation method in the cosmetic field.

### Preparation Example 6: Gel

| | |
|---|---|
| Phytoestrogen | 0.5 wt % |
| Sodium ethylenediamineacetate | 0.05 wt % |
| Glycerin | 5.0 wt % |
| Carboxyvinyl polymer | 0.3 wt % |
| Ethanol | 5.0 wt % |
| PEG60 hardened castor oil | 0.5 wt % |
| Triethanolamine | 0.3 wt % |
| Preservative, pigment, flavor | appropriate amounts |
| Purified water to 100 wt % | |

Although ingredients relatively suitable for gels are mixed in the above-described mixing ratio as an exemplary example, the mixing ratio may be arbitrarily modified, and the gel may be prepared according to a general preparation method in the cosmetic field.

Although ingredients relatively suitable for cosmetic compositions are mixed in the above-described mixing ratio as an exemplary example, these may be applied to cosmetics for various applications including color cosmetics, and may be used in the preparation of a medicine that can be applied thinly on the human body according to the efficacy thereof, and the mixing ratio may be arbitrarily varied depending on local and national preferences such as demand classes, demand countries, purposes of use, and the like.

1. A pharmaceutical composition for preventing hair loss or promoting hair growth, the pharmaceutical composition comprising, as an active ingredient, one or more selected from phytoestrogens represented by Formulae 1 to 4 below:

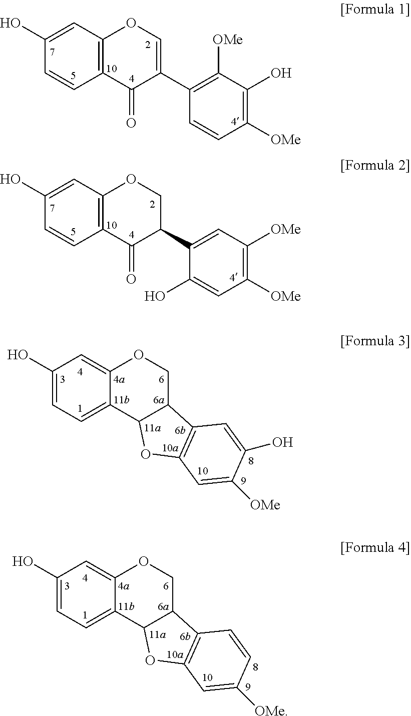 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

2. The pharmaceutical composition of claim 1, wherein the phytoestrogens are derived from a leguminous plant.

3. The pharmaceutical composition of claim 1, wherein the phytoestrogens increase the survival rate of human hair follicle dermal papilla cells.

4. The pharmaceutical composition of claim 1, wherein the phytoestrogens have an activity of elongating human hair length.

5. The pharmaceutical composition of claim 1, wherein the phytoestrogens are included in the composition at a concentration of 0.005 μM to 1 mM.

6. The pharmaceutical composition of claim 1, wherein the pharmaceutical composition is a preparation selected from the group consisting of a cream, a gel, a patch, a spray, an ointment, a plaster, a lotion, a liniment, a paste, and a cataplasma, the preparations being dermatologic agents.

7. A cosmetic composition for preventing hair loss or promoting hair growth, the cosmetic composition comprising, as an active ingredient, one or more selected from phytoestrogens represented by Formulae 1 to 4 below:

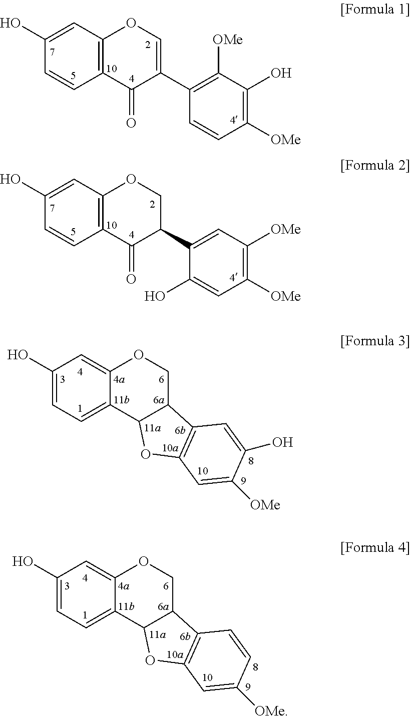 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

8. The cosmetic composition of claim 7, wherein the phytoestrogens are derived from a leguminous plant.

9. The cosmetic composition of claim 7, wherein the cosmetic composition is formulated into a hair tonic, a hair lotion, a hair cream, a hair spray, a hair mousse, a hair gel, a hair conditioner, a hair shampoo, a hair rinse, a hair pack, a hair treatment, an eyebrow growth agent, an eyelash growth agent, an eyelash nutrient, a shampoo for a pet, or a rinse for a pet.

10. A method of preventing or treating hair loss, or promoting hair growth, the method comprising administering a composition comprising, as an active ingredient, one or more selected from phytoestrogens represented by Formulae 1 to 4 below:

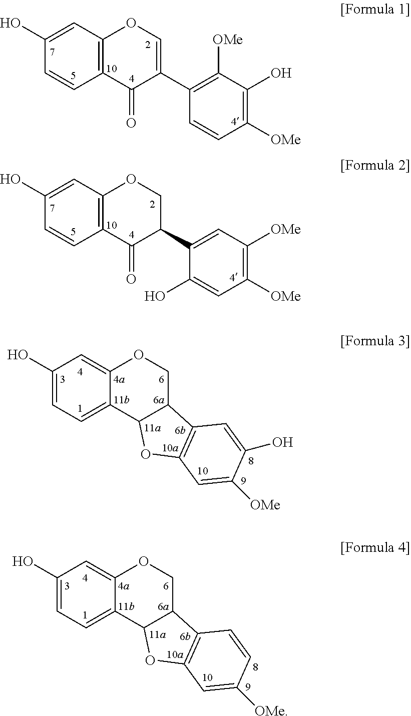 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]

11. The method of claim 10, wherein the phytoestrogens are derived from a leguminous plant.

12. A use of a composition comprising, as an active ingredient, one or more selected from phytoestrogens represented by Formulae 1 to 4 below, for preparing an agent for preventing or treating hair loss, or promoting hair growth: 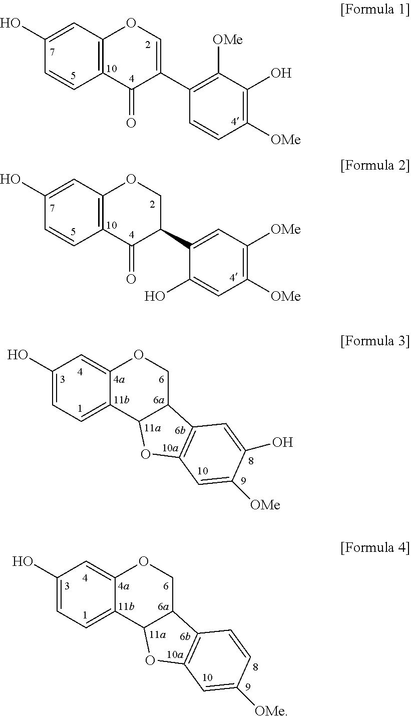 [SMILES: COc1ccc(-c2coc3cc(O)ccc3c2=O)c(OC)c1O ; COc1cc(O)c([C@@H]2COc3cc(O)ccc3C2=O)cc1OC ; COc1cc2c(cc1O)C1COc3cc(O)ccc3C1O2 ; COc1ccc2c(c1)OC1c3ccc(O)cc3OCC21]
